(19) United States
(12) Patent Application Publication
(10) Pub. No.: US 2009/0273768 A1
(43) Pub. Date: Nov. 5, 2009

(54) Liquid for immersion exposure, method of purifying the same,and immersion exposure method

(75) Inventors: Akifumi Kagayama, Chiba (JP); Takashi Nakano, Chiba (JP); Hiroaki Tamatani, Kanagawa (JP); Norio Nakayama, Chiba (JP)

Correspondence Address: BUCHANAN, INGERSOLL & ROONEY PC, POST OFFICE BOX 1404, ALEXANDRIA, VA, 22313-1404, US

(73) Assignee: Mitsui Chemicals, Inc., Tokyo (JP)

(21) Appl. No.: 11/919,245

(22) PCT Filed: Apr. 26, 2006
(86) PCT No.: PCT/JP2006/308722

§ 371 Date: Oct. 25, 2007

(30) Foreign Application Priority Data
Apr. 26, 2005 (JP) 2005 128504

(51) Int. Cl. G03B 27/32; C07C 7/12; C07C 9/00; C07C 13/00

(52) U.S. Cl. 355/77; 585/822; 585/16

(57) ABSTRACT

A saturated hydrocarbon compound is brought into contact at least with a first adsorbent and a second adsorbent to obtain a liquid for immersion exposure containing the saturated hydrocarbon compound with a purity of 99.5 wt % or more.

### TECHNICAL FIELD

The present invention relates to a liquid for immersion exposure, a method of purifying the liquid for immersion exposure, and an immersion exposure method, and more particularly, to a technique used for an immersion exposure apparatus, which interposes a liquid in a light path between a projection optical system and a substrate at the time of exposure using a projection exposure apparatus used for a lithography process for manufacturing various kinds of electronic devices including semiconductor integrated circuits.

### BACKGROUND ART

As patterns formed by a lithographic method become finer with an increase in integration and densification of various electronic devices, the most advanced process uses a 193 nm ArF laser that enables a line/space pattern width resolution of a half pitch of about 90 to 65 nm.

As a higher integration and densification of electronic devices is required, a finer lithographic process is required.

For the finer lithographic process, it is general to shorten a wavelength of exposure light. For a finer region under 65 nm node, an apparatus using an F₂ laser, an extreme ultraviolet (EUV), and so on are under development. However, there are many problems including a high price for developing optical lenses transparent to these wavelengths.

Another method for the finer lithographic process involves increasing a numerical aperture (NA) of a lens. It is common to increase the NA by enlarging an incident angle of the exposure light by a projection lens. However, this method has a problem of lowering a depth of focus (DOF) as well as a limit in the incident angle due to a difference in refractive index between the lens and the air.

An immersion exposure method has been proposed which increases an NA without lowering a DOF even for the same wavelength of exposure light using a conventional projection optical system (Patent Document 1).

This method is to interpose a liquid having a refractive index larger than that of air or gas such as nitrogen gas or the like at least a part of the region between the lens and a substrate. Assuming that the liquid has a refractive index of n, a wavelength of exposure light in the liquid decreases to 1/n of that in the air or nitrogen gas used in a conventional dry exposure method, thereby making it possible to further enlarge an incidence angle even for a light source having the same exposure wavelength, which may result in enhancement of resolution and hence further magnification of a DOF.

An immersion exposure method using pure water (refractive index of which is 1.44) as a high refractive index liquid and ArF laser as a light source can provide a line/space pattern resolution of a 45 nm half pitch, and various techniques related thereto have been already disclosed (Patent Document 2).

As a further finer range, a line/space pattern resolution of a 30 nm half pitch or so is required. It is required to use a liquid having a refractive index of more equal to or more than 1.6 at 193 nm wavelength in order to realize such a resolution with ArF immersion exposure. High transparency at a wavelength of 193 nm, specifically, more than 80% of transmittance for a 1 mm-thick film, are needed to maintain good exposure performance which is little effected by heat generated by a laser irradiation.

For an immersion exposure technique for providing a resolution of up to 45 nm under development, a fluorine solvent (Patent Document 3), which is examined equally with the pure water in order of materials with high transparency in a short wavelength region, is promising as a liquid having a refractive index higher than that of pure water. However, a structure containing fluorine has generally a low refractive index and a compound having a refractive index of 1.6 or more has not been discovered. In addition, examination using water added with an inorganic compound or using an organic solvent has been reported (Non-Patent Document 1 and Non-Patent Document 2). However, they also have problems as follows. That is, the water added with the inorganic compound, for example, an aqueous phosphoric acid solution, reaches a refractive index of 1.6 but has low transmittance and has a possibility to damage a lens or a substrate by the added compound. Among the organic solvents, alcoholic solvents such as glycerol (refractive index of which is 1.6) have a high refractive index but low transmittance due to adsorption around a wavelength of 190 nm.

Here, the liquid of high refractive index for the immersion exposure is designated as the next-generation of pure water and thus required to be supplied at low price. It is better if a recycle is possible at an actual site of the exposure (on site). For this purpose, a stable method of purification and re-purification by a simple facility is required.

On the other hand, when a liquid is purified for use in the immersion exposure, a degree of purification required for use in the immersion exposure should extremely suppress a degree of adsorption of light at a wavelength of 193 nm. However, since most organic materials have a large degree of adsorption of 250 nm or less, these impurities should be removed up to ppm order or below, whereby the purification is not easy.

A general example of a liquid purifying method is a distillation.

A method of purifying organic solvents by the use of silica gel to produce a solvent for spectrum measurement was known in the past as a simpler method (Non-Patent Document 3).

Patent Document 1: Japanese Patent Laid-Open No. 6-124873

Patent Document 2: Japanese Patent Laid-Open No. 2005-19616

Patent Document 3: Japanese Patent Laid-Open No. 2004-325466

Non-Patent Document 1: Proceedings of SPIE, 2004, Vol. 5377, pp 273-284

Non-Patent Document 2: International Symposium on Immersion and 157 nm Lithography, Aug. 2 to 5, 2004, Kapalan et al.

Non-Patent Document 3: Lecture on Experimental Chemistry, 5^{th} edition, Vol. 4, 2003, pp 71-72

### DISCLOSURE OF THE INVENTION

The purification by the distillation was an important method developed with various industrial techniques for a long time. However, the inventors found out that a precise distillation tower having a large number of theoretical plates is needed to satisfy the above-mentioned requirements for the liquid for immersion exposure with a high refractive index, thereby increasing cost and volume for the apparatus, so making it difficult to use it for the recycle on site. Since operating

A general example of a liquid purifying method is a distillation.

A method of purifying organic solvents by the use of silica gel to produce a solvent for spectrum measurement was known in the past as a simpler method (Non-Patent Document 3).

Patent Document 1: Japanese Patent Laid-Open No. 6-124873

Patent Document 2: Japanese Patent Laid-Open No. 2005-19616

Patent Document 3: Japanese Patent Laid-Open No. 2004-325466

Non-Patent Document 1: Proceedings of SPIE, 2004, Vol. 5377, pp 273-284

Non-Patent Document 2: International Symposium on Immersion and 157 nm Lithography, Aug. 2 to 5, 2004, Kapalan et al.

Non-Patent Document 3: Lecture on Experimental Chemistry, 5^{th} edition, Vol. 4, 2003, pp 71-72

### DISCLOSURE OF THE INVENTION

The purification by the distillation was an important method developed with various industrial techniques for a long time. However, the inventors found out that a precise distillation tower having a large number of theoretical plates is needed to satisfy the above-mentioned requirements for the liquid for immersion exposure with a high refractive index, thereby increasing cost and volume for the apparatus, so making it difficult to use it for the recycle on site. Since operating conditions for the distillation should be changed depending on a variation in quality of raw materials or variations of impurities due to a change in exposure condition on site, there remains a room to be improved for the stable purification.

In Non-Patent document 3 described above, as a purification result of n-pentane and cyclohexane, the transmittance at 220 nm increases which is a general lower limit for measuring an ultra violet visual spectrum, but the transmittance rapidly decreases in the range of shorter wavelength and is 80%/mm or less at a wavelength of 200 nm which is a lower limit of the measurement data.

The inventor keenly studied and found out that a material having high transmittance and a high refractive index at a wavelength of 193 nm can be obtained from saturated hydrocarbon compounds with high purity, and then completed the invention.

The invention provides a material having high transmittance and a high refractive index at a wavelength of an ArF laser as a liquid for immersion exposure.

That is, the invention relates to:

(1) a method of purifying a liquid for immersion exposure, wherein a saturated hydrocarbon compound is brought into contact with a first adsorbent and a second adsorbent to obtain a liquid for immersion exposure containing the saturated hydrocarbon conditions for the distillation should be changed depending on a variation in quality of raw materials or variations of impurities due to a change in exposure condition on site, there remains a room to be improved for the stable purification.

In Non-Patent document 3 described above, as a purification result of n-pentane and cyclohexane, the transmittance at 220 nm increases which is a general lower limit for measuring an ultra violet visual spectrum, but the transmittance rapidly decreases in the range of shorter wavelength and is 80%/mm or less at a wavelength of 200 nm which is a lower limit of the measurement data.

The inventor keenly studied and found out that a material having high transmittance and a high refractive index at a wavelength of 193 nm can be obtained from saturated hydrocarbon compounds with high purity, and then completed the invention.

The invention provides a material having high transmittance and a high refractive index at a wavelength of an ArF laser as a liquid for immersion exposure.

That is, the invention relates to:

(1) a method of purifying a liquid for immersion exposure, wherein a saturated hydrocarbon compound is brought into contact with a first adsorbent and a second adsorbent to obtain a liquid for immersion exposure containing the saturated hydrocarbon compound with a purity of 99.5 wt % or more;

(2) the method according to (1), further comprising bringing the saturated hydrocarbon compound into contact with the second adsorbent at the same time as bringing the saturated hydrocarbon compound into contact with the first adsorbent;

(3) the method according to (1), further comprising bringing the saturated hydrocarbon into contact with the second adsorbent after bringing the saturated hydrocarbon compound into contact with the first adsorbent;

(4) the method according to any one of (1) to (3), wherein the first adsorbent is activated carbon and the second adsorbent is silica gel or alumina;

(5) the method according to any one of (1) to (4), further comprising bringing the saturated hydrocarbon compound into contact with a third adsorbent or third to n^{th} (where n is an integer equal to or larger than 4) adsorbents;

(6) the method according to (5), wherein the first adsorbent is activated carbon, the second adsorbent is silica gel, and the third adsorbent is alumina;

(7) the method according to any one of (1) to (6), wherein the saturated hydrocarbon compound is bicyclohexyl;

(8) the method according to any one of (1) to (6), wherein the saturated hydrocarbon is a trans-decahydronaphthalene;

(9) an immersion exposure method, wherein the liquid for immersion exposure obtained by the method according to any one of (1) to (8) is filled in a space between a photosensitive material on a substrate and a projection lens and the photosensitive material is exposed thorough the liquid for immersion exposure;

(10) an immersion exposure method comprising: a step of degassing the liquid for immersion exposure obtained by the method according to any one of (1) to (8) and then supplying the degassed liquid to a space between a photosensitive material on a substrate and a projection lens; a step of exposing the photosensitive material through the liquid for immersion exposure; a step of recovering the liquid for immersion exposure after the step of exposing the photosensitive material; and a step of bringing the recovered liquid for immersion exposure into contact with at least one kind of adsorbent, wherein the liquid for immersion exposure is circulated between the space and the adsorbent;

(11) an immersion exposure method comprising: a step of obtaining a liquid for immersion exposure by the method according to any one of (1) to (8); a step of degassing the liquid for immersion exposure and then supplying the degassed liquid to a space between a photosensitive material on a substrate and a projection lens; a step of exposing the photosensitive material through the liquid for immersion exposure; a step of recovering the liquid for immersion exposure after the step of exposing the photosensitive material; and a step of bringing the recovered liquid for immersion exposure into contact again with the first adsorbent and the second adsorbent, wherein the liquid for immersion exposure is circulated between the space and the first or second adsorbent;

(12) a liquid for immersion exposure containing a saturated hydrocarbon compound with a purity of 99.5 wt % or more, obtained by the method according to any one of (1) to (8);

(13) the liquid for immersion exposure according to (12), wherein the saturated hydrocarbon compound has a linear or a branched chain structure and has a carbon number of 12 or more;

(14) the liquid for immersion exposure according to (12), wherein the saturated hydrocarbon compound has a structure including a cyclic backbone and has a carbon number of 7 or more; and

(15) an immersion exposure method using the liquid for immersion exposure according to any one of (12) to (14).

### BRIEF DESCRIPTION OF THE DRAWINGS

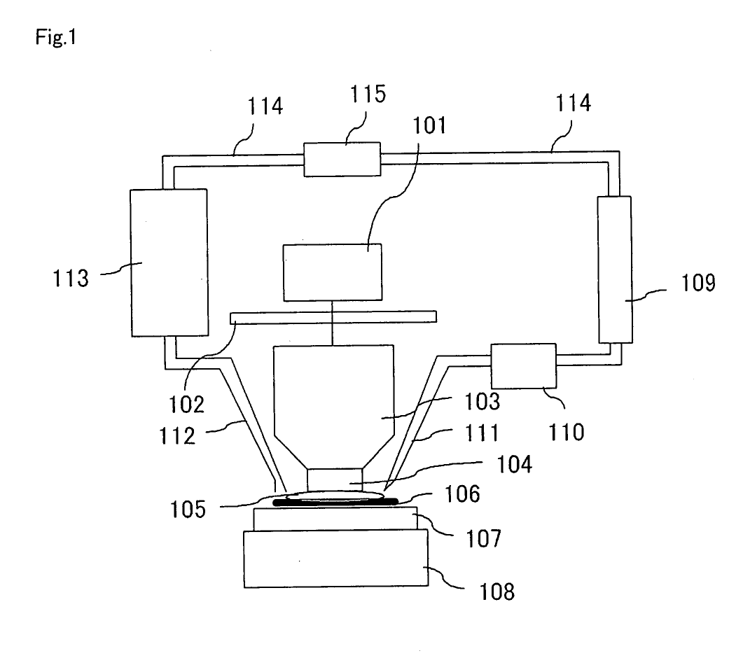

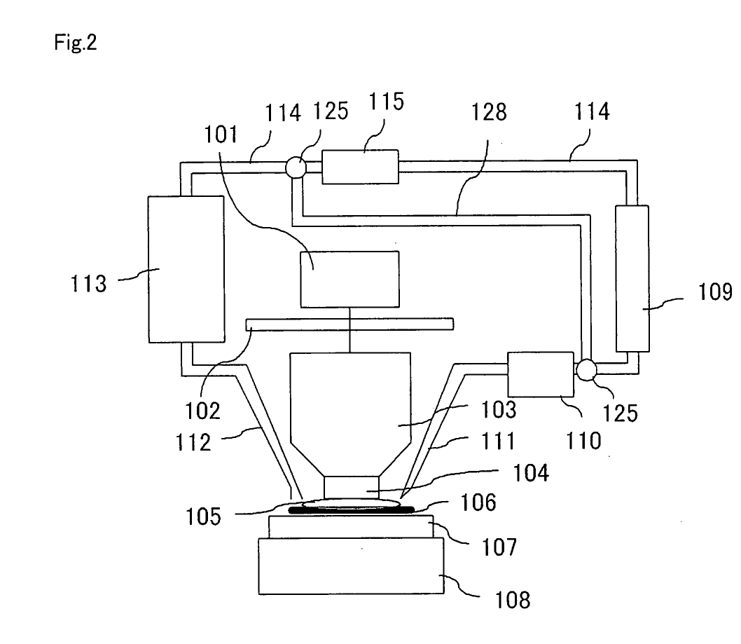

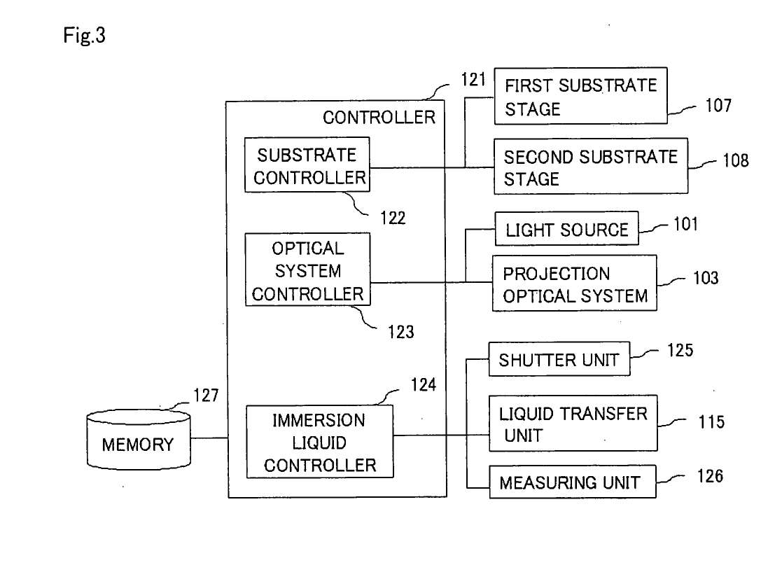

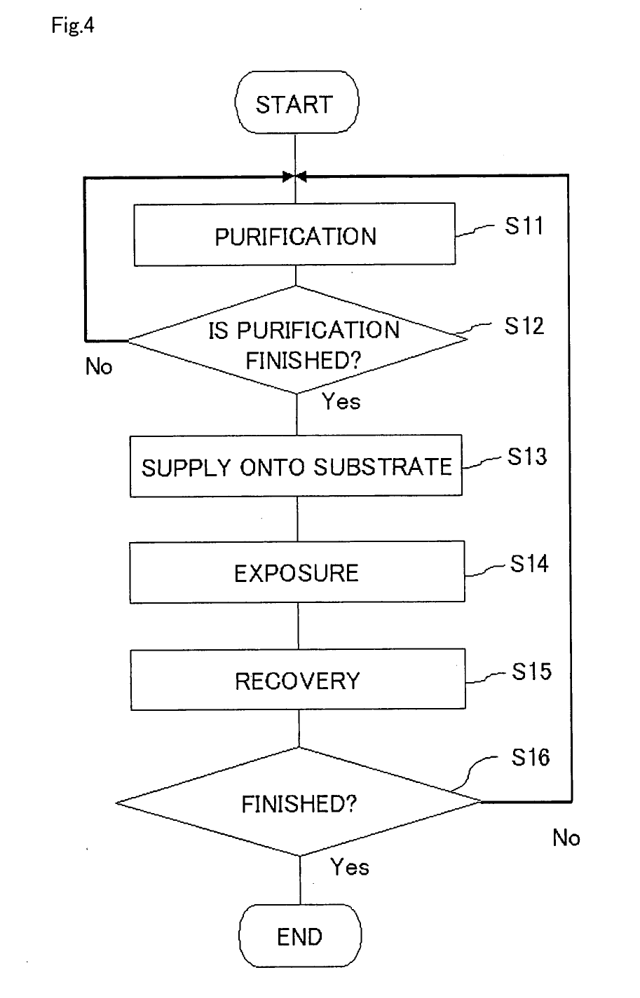

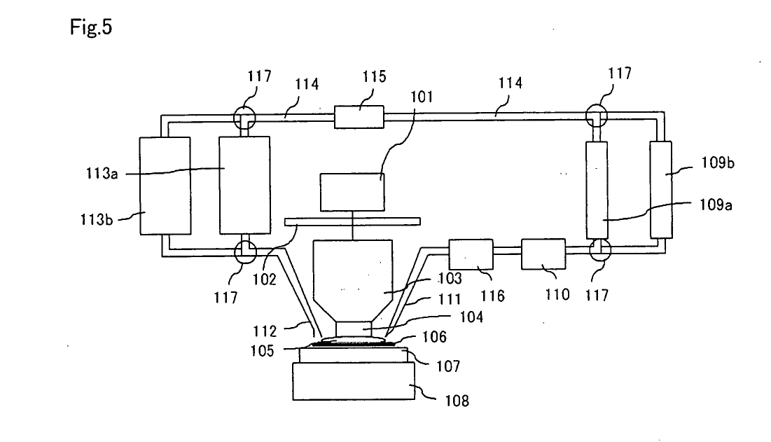

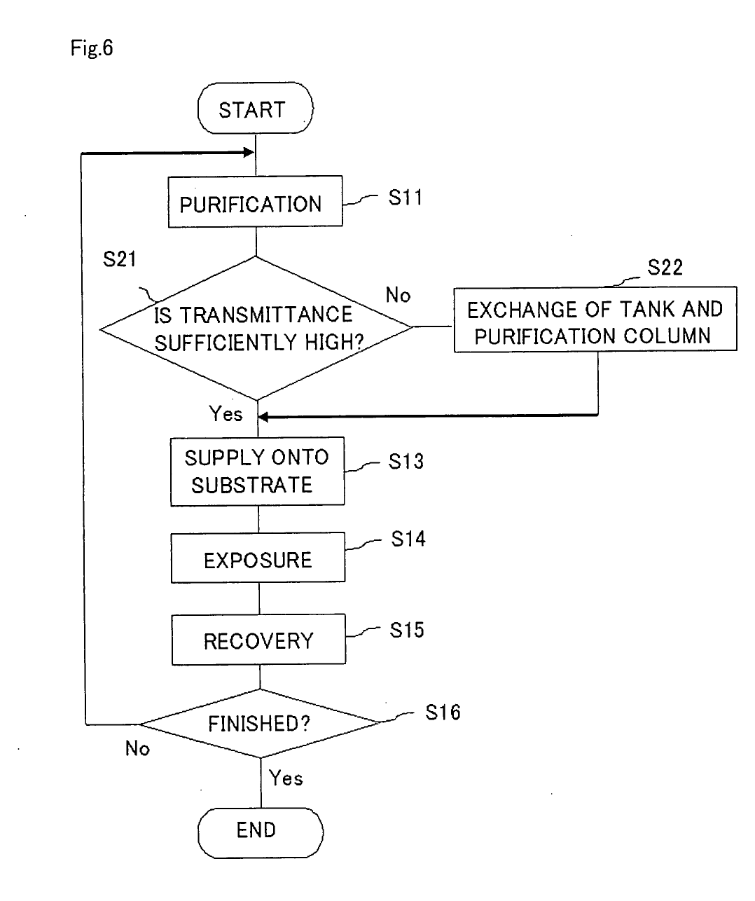

The above and other objects, features and advantages described in the above will be more apparent from the preferred embodiments given below and accompanying drawings.

[FIG. 1] A diagram illustrating a configuration of an immersion exposure apparatus of an embodiment.

[FIG. 2] A diagram illustrating a configuration of an immersion exposure apparatus of an embodiment.

[FIG. 3] A functional block diagram illustrating a configuration of an immersion exposure apparatus of an embodiment.

[FIG. 4] A flowchart illustrating an order of exposure of an embodiment.

[FIG. 5] A diagram illustrating a configuration of an immersion exposure apparatus of an embodiment.

[FIG. 6] A flowchart illustrating an order of exposure of an embodiment.

### BEST MODE FOR CARRYING OUT THE INVENTION

In a liquid for immersion exposure according to the invention, a purity of a saturated hydrocarbon compound is 99.5 wt % or more. By raising the purity to 99.5 wt % or more, it is possible to obtain a liquid with a high refractive index, in which the transmittance is 80%/mm or more, preferably 90%/mm or more, and more preferably 98%/mm or more at a wavelength of 193 nm. Accordingly, the obtained liquid can be properly used as an immersion exposure medium.

The purity of the saturated hydrocarbon compound means a ratio of the saturated hydrocarbon compound to the entire liquid for immersion exposure. The saturated hydrocarbon compound may include a single kind or plural kinds. When the saturated hydrocarbon compound includes the plurality of kinds, the purity is a ratio of all the included saturated hydrocarbon compounds to the entire liquid for immersion exposure. In view of further raising the transmittance at a wavelength of 193 nm, the purity of the saturated hydrocarbon compound according to the invention is preferably 99.9 wt % or more.

The liquid for immersion exposure according to the invention has a refractive index of 1.5 and more preferably 1.6 or more, in view of further raising a resolution.

The saturated hydrocarbon compound used as the liquid for immersion exposure in the invention is not particularly limited but is specifically described below.

The linear- or branched-chain compounds are expressed by a molecular formula, CₙH₂ₙ₊₂ (where n is a natural number, which is true in the following description), where n is preferably 12 or more, and examples thereof include dodecanes, tridecanes, tetradecanes, pentadecaned, and hexadecanes such as n-dodecane, 2-methylundecane, 3-ethyldecane, and 4-propylnonane.

The compounds having a cyclic backbone may include a monocyclic structure or polycyclic structure, may have a substituent of a linear chain or a branched chain, and are expressed by molecular formulas such as CₙH₂ₙ (mono-cyclic), CₙH₂ₙ₋₂ (bi-cyclic), and CₙH₂ₙ₋₄ (tri-cyclic). Here, n is preferably 7 or more. Examples of the mono-cyclic compounds include cycloheptane and cyclodecane, examples of the bi-cyclic compounds include octahydroindene, bicyclohexyl, decahydronaphthalene, and norborane, and examples of the tri-cyclic compounds include dodecahydrofluorene and tetradecahydrophenanthrene.

These saturated hydrocarbon compounds may be used independently or in combinations of a plurality of compounds.

Since the saturated hydrocarbon compound used in the invention has high stability against light, heat, oxygen, and so on and has small corrosiveness, it can be easily treated and can be industrially purchased or synthesized at a low price. Accordingly, the compound can be applied to the immersion exposure technique using pure water, which is currently under development, without a large technical modification or cost.

Therefore, the liquid for immersion exposure according to the invention can realize finer resolution by the use of the conventional exposure apparatus is possible. Especially, since it is possible to easily form around 30 nm line/space pattern required for manufacturing, for example, next-next-generation electronic devices by applying the liquid for immersion exposure according to the invention to an ArF immersion exposure apparatus, the invention is valuable for industries.

As mentioned in the section of BACKGROUND ART, when purifying the liquid for immersion exposure, a simple method with a high purity is required.

Thus, the inventor studied hard the methods of purifying the liquid for immersion exposure. As a result, the inventor found out that the liquid for immersion exposure including the saturated hydrocarbon compound with a purity of 99.5 wt % or more can be simply obtained by bringing the saturated hydrocarbon compound with different kinds of adsorbents, thereby obtaining high transmittance.

When a plurality of impurities is included in the saturated hydrocarbon compound, it is important to reduce concentrations of the impurities below a predetermined level so as to raise the purity, transmittance, and refractive index of the saturated hydrocarbon compound. Even when plural impurity components to be necessarily removed exist in the saturated hydrocarbon compound and it is difficult to remove all the impurity components by the use of one adsorbent, the impurity components having different features can be efficiently removed by using combinations of plural adsorbents. Accordingly, the purity of the saturated hydrocarbon compound can be further enhanced.

Hereinafter, the method of purifying the liquid for immersion exposure using plural adsorbents will be described in more detail.

According to the invention, the saturated hydrocarbon compound is brought into contact with first and second adsorbents to obtain a liquid for immersion exposure including the saturated hydrocarbon compound with a purity of 99.5 wt % or more. The step of bringing the saturated hydrocarbon compound into contact with the first adsorbent and the step of bringing the saturated hydrocarbon compound into contact with the second adsorbent may be the same step or different steps. The second adsorbent may serve as a filter having a filtering function of physically separating micro particles included in the liquid or the first adsorbent from each other.

For example, the first adsorbent and the second adsorbent may be mixed and brought into contact with the saturated hydrocarbon compound so as to bring the saturated hydrocarbon compound into the first adsorbent and the second adsorbent at the same time. The first adsorbent and the second adsorbent may be received in different spaces so as to perform the step of bringing the saturated hydrocarbon compound into contact with the second adsorbent after the steps of bringing the saturated hydrocarbon compound into contact with the first adsorbent.

The contact with the adsorbents may be performed by a batch method or a column chromatography. The contact with the adsorbents may be performed at a single time or at multiple times.

Combinations of plural kinds selected depending on the natures of the saturated hydrocarbon compound can be used as the adsorbent and examples thereof include silica gel, activated carbon, alumina (activated alumina), zeolite, and molecularsieve.

A specific combination of the adsorbent may include a combination in which the first adsorbent is activated carbon and the second adsorbent is silica gel or alumina. Accordingly, the purity and the transmittance of the saturated hydrocarbon compound can be certainly further enhanced.

The adsorbents have, for example, a particle shape. Thus, it is possible to easily fill a predetermined region of a supply system of the liquid for immersion exposure in an exposure apparatus with the adsorbents and to increase specific surface areas of the adsorbents.

The method of purifying the liquid for immersion exposure may include a step of bringing the saturated hydrocarbon compound into contact with a third adsorbent or third to n^{th} (where n is an integer equal to or larger than 4) adsorbents. In this case, plural impurities can be more efficiently removed even when the plural impurities are included in the saturated hydrocarbon compound.

The step of bringing the saturated hydrocarbon compound into contact with the third adsorbent or the third to n^{th} (where n is an integer equal to or larger than 4) adsorbents and the step of bringing the saturated hydrocarbon compound into contact with the first or second adsorbent may be the same step or separate steps. The third to n^{th} adsorbents may serve as a filter having a filtering function of physically separating micro particles and the adsorbents included in the liquid from each other.

An example of the step of brining the saturated hydrocarbon compound into contact with the third adsorbent is the same as the step of bringing the saturated hydrocarbon compound with the first or second adsorbent may be a method of bringing the saturated hydrocarbon compound into contact with a mixture of the first, second, and third adsorbents. It may be also allowable to bring the saturated hydrocarbon compound into contact with the second adsorbent after bringing the saturated hydrocarbon compound with a mixture of the first and third adsorbents.

An example of the step of bringing the saturated hydrocarbon compound into contact with the third adsorbent is a step different from the step of bringing the saturated hydrocarbon compound into contact with the first or second adsorbent may be a method of bringing the saturated hydrocarbon compound into contact with the first, second, and third adsorbents received in different spaces in a predetermined order. More specifically, in a state where the activated carbon is used as the first adsorbent, silica gel is used as the second adsorbent, and alumina is used as the third adsorbent, it is possible to bring the saturated hydrocarbon compound into contact with the first, second, and third adsorbents in this order.

In case of contact with four or more kinds of adsorbents, predetermined adsorbents may be properly combined similarly to the case of contact with three or less kinds of adsorbents.

Specifically, an example of a purification apparatus for manufacturing the liquid for immersion exposure according to the invention may include an apparatus with a raw material tank in which a raw liquid and the first adsorbent are charged and stirred, then allowing the liquid to pass through a column filled with the second adsorbent, and reserving the liquid as the immersion liquid in a reserving tank. As mentioned above, it is possible to allows the liquid to continuously pass through columns filled with the second, the third, and the n^{th} (where n is an integer equal to or larger than 4) adsorbents. One column may be filled with plural kinds of adsorbents. A circulating system of allowing the liquid to pass again through the columns having adsorbents, when the liquid passing through the column is sampled, the purity thereof is measured by a gas chromatography method or a transmittance spectrum method and the like, and the purity of the sampled liquid is not 99.5 wt % or more or equal to or greater than predetermined transmittance, may be taken.

Next, an exposure method using the liquid for immersion exposure according to the invention will be described with reference to the drawings. In this method, the space interposed between a photosensitive material on a substrate and a projection lens is filled with the liquid for immersion exposure obtained by the purification method using an adsorbent and the photosensitive material is exposed through the liquid for immersion exposure. All the common elements are denoted by the same reference numerals and description thereof is properly not repeated.

FIG. 1 is a diagram illustrating a configuration of an immersion exposure apparatus according to an embodiment of the invention.

In the exposure apparatus shown in FIG. 1, light emitted from a light source 101 for exposure is radiated onto a surface of a substrate 106 through a mask 102, a projection optical system 103, a projection lens 104, and an immersion liquid 105. The mask 102 is disposed between the light source 101 and the projection optical system 103. An image of the mask 102 is projected onto the substrate 106 and exposed to light.

The substrate 106 is a semiconductor wafer such as a silicon wafer on which a photoresist film is formed. The substrate 106 is disposed on a first substrate stage 107.

The first substrate stage 107 is provided on a second substrate stage 108. These substrate stages serves to move and fix the substrate 106. One substrate stage may be an XY stage and the other substrate stage may be a Z stage, thereby constituting two steps.

The immersion liquid 105 is the liquid for immersion exposure according to the invention. The immersion liquid 105 is supplied to a region interposed between the substrate 106 and the projection optical system 103.

The apparatus shown in FIG. 1 includes a circulation supply system for the immersion liquid 105.

As for the supply system for the immersion liquid 105, a liquid reservoir 113 for receiving the immersion liquid 105, a liquid transfer unit 115 for supplying the immersion liquid 105 in the liquid accumulating bath 113 downstream, a liquid purification unit 109 for removing impurities in the immersion liquid 105, and a degassing unit for degassing the immersion liquid 105 are connected. A liquid circulating pipe 114 is connected between the liquid reservoir 113 and the liquid transfer unit 115 and between the liquid transfer unit 115 and the liquid purification unit 109.

The liquid purification unit 109 may be disposed at any position between a liquid recovering pipe 112 and the degassing unit 110, may be provided in a single or plural, or may be formed integrally with the liquid reservoir 113.

Among a circulating path of the immersion liquid 105 from the liquid recovering pipe 112 to the liquid supply pipe 111, two or more kinds of adsorbents are disposed in a region from the liquid recovering pipe 112 to the liquid purification unit 109. However, when the saturated hydrocarbon compound purified by bringing the saturated hydrocarbon compound into contact with the two or more kinds of adsorbents is purified again after being exposed, one kind of adsorbent may be provided in a region from the liquid recovering pipe 112 to the liquid purification unit 109.

In the initial state, a first adsorbent is placed and agitated, for example, in a raw material bath filled with the above-mentioned raw liquid, then the liquid is made to pass through a column filled with a second adsorbent, and the saturated hydrocarbon compound sufficiently purified in advance or a commercial saturated hydrocarbon compound is received in the reservoir for reserving an immersion liquid. The purity of the commercial saturated hydrocarbon compound is preferably 60 wt % or more, more preferably 80 wt % or more, and still more preferably 95 wt % or more. The immersion liquid 105 is supplied from the liquid accumulating bath 113 to the liquid purification unit 109 in the course of passing through the circulating system before it is supplied to the substrate 106. Accordingly, the impurities in the immersion liquid 105 are removed to enhance the purity. The saturated hydrocarbon compound is purified and degassed by the degassing unit 110. The degassed liquid is supplied as the immersion liquid 105 to the substrate 106 via the liquid supply pipe 111 and is filled in the region between the projection lens 104 and the substrate.

The immersion liquid 105 after the exposure is recovered into the liquid recovering pipe 112 and is accumulated again in the connected liquid reservoir 113 via the liquid purification unit as needed.

The liquid purification unit 109 is a column filled with, for example, a predetermined adsorbent. The liquid purification unit 109 may be filled with one kind of adsorbent or may be filled with plural kinds of adsorbents.

The liquid purification unit 109, the liquid accumulating bath 113 or the liquid recovering pipe 112, and the liquid circulating pipe 114 may be filled with different kinds of adsorbents. More specifically, the liquid reservoir 113 or the liquid circulating pipe 114 may be filled with activated carbon and the liquid purification unit 109 may be filled with silica gel. The liquid reservoir 113 or the liquid circulating pipe 114 may be filled with activated carbon and the liquid purification unit 109 may be filled with alumina and silica gel.

The kinds, the amounts, and the arrangements of the adsorbents may be determined in the following order. The liquid accumulating bath 113, the liquid circulating pipe 114, or the liquid purification unit 109 is first filled with a predetermined adsorbent and then the liquid is made to move from the liquid reservoir 113 to the liquid purification unit 109. Then, the liquid having passed through the liquid purification unit 109 is sampled and the purity is measured by the gas chromatography method or the transmittance spectrum method and so forth. Additionally, the kinds, the amounts, and the arrangements of the adsorbents are determined so that the sampled immersion liquid 105 has a purity of 99.5 wt % or more or has transmittance equal to or greater than a predetermined value.

According to this embodiment, since a supply path of the immersion liquid 105 in the exposure apparatus constitutes a circulating system, the immersion liquid 105 can be repeatedly used. Since the liquid purification unit 109 is disposed in the circulating path, it is possible to simply and efficiently purify the immersion liquid 105 on site, thereby using the saturated hydrocarbon compound with a high purity as an exposure medium. By embodying the liquid purification unit 109 as a column filled with an adsorbent, it is possible to simply and satisfactorily enhance the purity of the saturated hydrocarbon compound.

FIG. 2 is a diagram illustrating another configuration of an immersion exposure apparatus according to an embodiment. The exposure apparatus shown in FIG. 2 also includes a circulating system for the immersion liquid 105 and has the same basic configuration as shown in FIG. 1. However, in this apparatus, the first and second adsorbents are filled in the liquid purification unit 109 and the liquid reservoir 113, respectively, and a connection pipe 128 and a shutter unit 125 are further provided. Similar to FIG. 1, a combination of the liquid purification unit 109, the shutter unit 125, and the connection pipe 128 may be disposed at any position between the liquid recovering pipe 112 and the degassing unit 110, may be in a single or plural, or may be formed integrally with the liquid reservoir 113. Any case will not cause inconvenience in the following description.

The connection pipe 128 is connected to a predetermined position on the downstream side of the liquid purification unit 109 and a predetermined position of the liquid circulating pipe 114. The shutter unit 125 is respectively disposed at both ends of the connection pipe 128. The shutter unit 125 is disposed at a position where the liquid circulating pipe 114 communicates with another pipe, is a member for adjusting the moving direction of the immersion liquid 105, and is embodied as, for example, a three-way cork.

FIG. 3 is a functional block diagram illustrating a configuration of the immersion exposure apparatus shown in FIG. 2. The exposure apparatus shown in FIG. 2 further includes a controller 121, a memory 127, and a measuring unit 126, as shown in FIG. 3.

The controller 121 includes a substrate controller 122, an optical system controller 123, and an immersion liquid controller 124.

The substrate controller 122 controls the position of the substrate 106 so as to, for example, control the movements of the first substrate stage 107 and the second substrate stage 108.

The optical system controller 123 controls operations of the optical system such as the light source 101 and the projection optical system 103.

The immersion liquid controller 124 controls the movement of the immersion liquid 105 by controlling operations of the shutter unit 125, the liquid transfer unit 115, and the measuring unit 126, for example.

The measuring unit 126 measures, for example, the purity of the immersion liquid 105. The measuring unit 126 may measure the transmittance of the liquid at 193 nm. The measuring unit 126 is disposed, for example, at a predetermined position between the liquid purification unit 109 and the degassing unit 110.

The memory 127 stores data of a critical value (lower limit) measured by the measuring unit 126 and stores data such as the purity, the transmittance, and the refractive index of the immersion liquid 105.

FIG. 4 is a flowchart illustrating an exposure sequence of the exposure apparatuses shown in FIGS. 2 and 3. Hereinafter, the exposure sequence using the exposure apparatuses shown in FIGS. 2 and 3 is described in more detail with reference to FIG. 4.

First, the saturated hydrocarbon compound in the liquid reservoir 113 is purified (S11).

In a viewpoint of a reliable enhancement in purity and light transmittance of the saturated hydrocarbon compound due to the purification with two or more kinds of adsorbents, the raw material purity of the saturated hydrocarbon compound in the liquid reservoir 113 is preferably 60 wt % or more, more preferably 80 wt % or more, and still more preferably 95 wt % or more.

In step 11, the immersion liquid controller 124 controls the operations of the shutter unit 125 and the liquid transfer unit 115 to move the liquid in the liquid reservoir 113 to the liquid purification unit 109 via the liquid circulating pipe 114. In the course of the movement, the liquid sequentially comes in contact with the first adsorbent filled in the liquid reservoir 113 and the second adsorbent filled in the liquid purification unit 109.

The liquid having passed through the liquid purification unit 109 is provided for the measurement of the measuring unit 126. The immersion liquid controller 124 acquires measured data obtained from the measuring unit 126. The immersion liquid controller 124 acquires data of the threshold transmittance value of the immersion liquid 105 with reference to the memory 127 and compares the acquired data with the data measured by the measuring unit 126.

When the measured data obtained from the measuring unit 126 is less than the critical value (No in step S12), the immersion liquid controller 124 controls the operation of the shutter unit 125 to return the liquid having passed through the liquid purification unit 109 to the liquid circulating pipe 114 through the connection pipe 128. Then, at least a part of the purification process is repeated. In FIG. 2, the liquid comes in contact with the adsorbent in the liquid purification unit 109 by passing through again the liquid purification unit 109.

When the measured data obtained from the measuring unit 126 is equal to or greater than the critical value (Yes In step S12), the purification process is finished. The immersion liquid controller 124 controls the operation of the shutter unit 125 to introduce the liquid for immersion exposure obtained in the purification process into the degassing unit 110 and supplies the degassed liquid to fill the space interposed between the photosensitive material (photoresist) on the substrate 106 and the projection lens (projection lens 104) from the liquid supply pipe 111 (S13).

At least at the time of exposure, the oxygen concentration in the immersion liquid 105 is preferably as low as possible. When oxygen exists, the transmittance may be decreased because of absorption of dissolved oxygen itself or ozone or oxide produced by laser irradiation. When gas is dissolved with a high concentration, bubbles may be easily produced in the liquid, thereby causing faults at the time of exposure. Accordingly, the circulating path is preferably in the atmosphere of nitrogen or an inert gas and preferably performs a degassing process just before the exposure.

The photoresist is exposed through the immersion liquid (S14). In step S14, the optical system controller 123 controls the operation of the optical system and the immersion liquid controller 124 controls the position of the substrate 106 by moving the first substrate stage 107 and the second substrate stage 108.

After the exposure of step 14, the immersion liquid controller 124 recovers the immersion liquid 105 to the liquid reservoir 113 through the liquid recovering pipe 112 (S15). When the exposure process is performed again (No in step S16), the liquid recovered to the liquid reservoir 113 is purified again by bringing the liquid into contact with the first and second adsorbents (S11). In this way, in this embodiment, the immersion liquid 105 is circulated between the space between a coating layer covering the photoresist or a resist and the lens, and the first and second adsorbent. The liquid for immersion exposure is repeatedly purified and used for the exposure.

In the above-mentioned sequence, it is possible to purify and recycle the immersion liquid 105 on site by the use of a simple method and to further enhance the purity of the immersion liquid 105 and the transmittance at 193 nm to a value greater than the desired value.

FIG. 5 is a diagram illustrating a configuration of an immersion exposure apparatus according to another embodiment of the invention. FIG. 6 is a flowchart illustrating an exposure sequence using the immersion exposure apparatus shown in FIG. 5.

The immersion exposure apparatus shown in FIG. 5 has the same basic configuration as the apparatus described with reference to FIG. 1. However, while one liquid purification unit 109 is disposed in FIG. 1, two of a first liquid purification unit 109a and a second liquid purification unit 109b are disposed in FIG. 5 in parallel to be switched to the circulating path of the immersion liquid 105. In FIG. 5, two of a liquid reservoir 113a and a liquid reservoir 113b are disposed in parallel to be switched to the circulating path of the immersion liquid 105.

A switching valve 117 for switching the moving path of the immersion liquid 105 is provided between the liquid recovering pipe 112 and the liquid reservoirs 113a and 113b and between the liquid circulating pipe 114 and the liquid reservoirs 113a and 113b. For example, the controller 121 controls the operation of the switching valves 117 to move the liquid recovered from the liquid recovering pipe 112 to any one of the liquid reservoir 113a and the liquid reservoir 113b.

Similarly, the switching valves 117 are also disposed between the liquid circulating pipe 114 and the liquid purification units 109a and 109b and between the degassing unit 110 and the liquid purification units 109a and 109b. For example, the controller 121 controls the operation of the switching valves 117 to move the immersion liquid 105 to any one of the liquid purification unit 109a and the liquid purification unit 109b. Similarly to FIG. 1, a combination of a plurality of the liquid purification units 109 and the switching valves 117 may be disposed at any position between the liquid recovering pipe 112 and the degassing unit 110, may be in a single or plural, or may be formed integrally with the liquid accumulating bath 113. Any case will not cause inconvenience in the following description.

In addition, in FIG. 5, a transmittance measuring unit 116 for measuring the transmittance of the immersion liquid 105 is disposed between the degassing unit 110 and the liquid supply pipe 111.

The first adsorbent is disposed in the liquid accumulating bath 113 or the liquid circulating pipe 114. The second adsorbent is disposed in the liquid purification unit 109. The second adsorbent may be disposed in the liquid recovering pipe 112 so as to remove impurities in the liquid recovered from the liquid recovering pipe 112 just in front of the liquid reservoir 113a or the liquid reservoir 113b.

A single or plural liquid transfer units 115 are installed in any one of the liquid circulating pipe 114, the liquid supply pipe 111, and the liquid recovering pipe 112 as needed.

The circulating path of the immersion liquid 105 is filled with the immersion liquid as much as possible and a remaining gas-phase portion is in the atmosphere of nitrogen or an inert gas.

In FIGS. 5 and 6, plural liquid reservoirs 113 and liquid purification units 109 are provided. In the initial state, the liquid reservoir 113a or the liquid reservoir 113b is filled with the first adsorbent purified or activated in advance and the liquid purification unit 109a and the liquid purification 109b are filled with the second adsorbent purified or activated in advance.

The raw material purity of the saturated hydrocarbon compound is preferably 60 wt % or more, more preferably 80 wt % or more, and still more preferably 95 wt % or more. The first adsorbent is placed and agitated, for example in a raw material tank filled with the above-mentioned raw liquid, the agitated liquid is made to pass through a column filled with the second adsorbent, and the liquid sufficiently purified in advance by an apparatus for reserving the immersion liquid in the reservoir is filled in the liquid reservoir 113. When the liquid is purified again and recycled after the exposure, only one kind of adsorbent may be used.

At the time of exposure, the immersion liquid 105 is continuously circulated and when the transmittance of the liquid having been subjected to the purification process (S11) and being measured by the transmittance measuring unit 116 is equal to or greater than the critical value (Yes in step S21), the immersion liquid 105 is supplied onto the substrate 106 (S13). Similarly to the apparatus shown in FIGS. 1 and 2, the exposure process (S14) and the recovery process (S15) of the immersion liquid 105 are performed. On the other hand, in the step where the transmittance is less than the predetermined critical value (No in step S21), the plural switching valves 117 are simultaneously and instantaneously operated to switch the moving path of the immersion liquid 105 and exchanges the used liquid purification unit 109 and the liquid reservoirs 113 (S22). Since the plural liquid reservoirs 113 and the plural liquid purification units 109 are provided, it is possible to perform a continuous operation without stopping the circulation of the immersion liquid 105.

Although two liquid purification units 109 and two liquid reservoirs 113 are provided in FIG. 5, the number of liquid purification units 109 and the number of liquid reservoirs 113 are not particularly limited and three or more units or baths may be provided.

According to the invention, a high-refractive transparent liquid having the transmittance equal to and the refractive index higher than those of pure water is provided which can enable a resolution process finer than the pure water by applying the liquid to the existing immersion exposure apparatus. Accordingly, the liquid can be used for manufacturing electronic devices with high integration and density.

In the method of purifying the saturated hydrocarbon compound according to the invention, the saturated hydrocarbon is brought into contact with at least the first and second adsorbents to have a purity of 99.5 wt % or more and a different purification method or a new synthesis method may be combined as needed when the purity of a raw material is low. The purification method and the synthesis method are not particularly limited, but the commercial product may be purified by the use of a distillation method in addition to the column chromatography method using activated carbon or silica gel. In an example of the new synthesis method, a compound having the same carbon backbone and an unsaturated bond may be reduced with hydrogen and synthesized and may be purified in the same was as described above.

In the first purification of the invention, the saturated hydrocarbon compound with a purity of 99.5 wt % or more is obtained by bringing the raw material into contact with at least the first and second adsorbents. However, the saturated hydrocarbon compound can be brought into contact with at least one kind of adsorbent when the saturated hydrocarbon compound having been purified by the above-mentioned method is purified and used again for exposure after being used in the exposure. For example, in FIG. 4 or 6, the immersion liquid 105 is recovered (S15) after step 14 of exposing the photoresist is performed, and the immersion liquid 105 is supplied again to the substrate 106 after a single-stage or plural-stage step of bringing the recovered immersion liquid 105 into contact with at least one kind of adsorbent is performed. In this way, it is possible to circulate the immersion liquid 105 between the adsorbents and the space between the substrate 106 and the projection lens 104.

### EXAMPLES

The invention will be described in more detail with reference to the following examples. The invention is not limited to the following examples.

In the following examples and comparative examples, the purity of a liquid was quantitated by a gas chromatography method (column: SUPELCO EQUITY-1; inner diameter of 0.25 mm; length of 60 m; membrane thickness of 0.25 μm; temperature of 40° C. to 300° C.; temperature rising rate of 10° C./min; Flame Ionization Detector (FID)). The contents of the micro impurities existing by 0.1 wt % or more were checked by the use of a combination of a particular gas chromatography and a mass spectrum analysis.

The transmittance was measured in a transmittance measuring mode using a cell, which is obtained by placing a sample into a capped quartz cell with a light path length of 10 mm, bubbling the quartz cell with nitrogen for 30 minutes or more, and then filling the cell with nitrogen, as a reference and using an ultraviolet visible spectrum photometer (U-3010, manufactured by Hitachi). Refractive indexes were measured by a minimum declination method with a Goniormeter spectrometer (type 1 UV-VIS-IR, manufactured by German MOLLER-WEDEL Co.). A wavelength for measuring the transmittance and the refractive indexes was 193.4 nm at 23° C.

The following adsorbents were used in the following examples and comparative examples.

Silica gel; Waco gel C-200, manufactured by Waco Pure Chemical

Alumina: Alumina A, Super-I, manufactured by ICN Co.

Activated carbon: RO, pellet, manufactured by Norit Co.

### Example 1

A highly-refractive liquid having a purity of 99.9 wt % or more, transmittance 97%/mm and a refractive index 1.64 at a wavelength of 193 nm was obtained by adding 1 part by weight of activated carbon to 10 parts by weight of trans-decahydronaphthalene, stirring the mixture for 24 hours at a room temperature, and then filtering the stirred mixture by using 1 part by weight of silica gel.

### Example 2

A highly-refractive liquid having a purity of 99.9 wt % or more, transmittance 90%/mm, and a refractive index of 1.65 at a wavelength of 193 nm was obtained by adding 1 part by weight of activated carbon to 10 parts by weight of cis-decahydronaphthalene, stirring the mixture for 24 hours at a room temperature, and then filtering the stirred mixture by using 1 part by weight of silica gel.

### Example 3

A highly-refractive liquid having a purity of 99.9 wt % or more, transmittance of 92%/mm, and a refractive index of 1.61 at a wavelength of 193 nm was obtained by adding 1 part by weight of activated carbon to 10 parts by weight of cyclooctane, stirring the mixture for 24 hours at a room temperature, and filtering the stirred mixture by using 1 part by weight of silica gel.

### Example 4

A highly-refractive liquid having a purity of 99.9 wt % or more, transmittance of 91%/mm, and a refractive index of 1.60 at a wavelength of 193 nm was obtained by adding 1 part by weight of activated carbon to 10 parts by weight of 2,3,10-tri methyl dodecane, stirring the mixture for 24 hours at a room temperature, and then filtering the stirred mixture by using 1 part by weight of silica gel.

### Example 5

A highly-refractive liquid having a purity of 99.9 wt % or more, transmittance 90%/mm, and a refractive index of 1.52 at a wavelength of 193 nm was obtained by adding 1 part by weight of activated carbon to 10 parts by weight of n-heptane, stirring the mixture for 24 hours at a room temperature, and then filtering the stirred mixture by using 1 part by weight of silica gel.

### Example 6

A highly-refractive liquid having a purity of 99.9 wt % or more, transmittance 90%/mm, and a refractive index of 1.56 at a wavelength of 193 nm was obtained by adding 1 part by weight of activated carbon to 10 parts by weight of cyclohexane, stirring the mixture for 24 hours at a room temperature, and then filtering the stirred mixture by using 1 part by weight of silica gel.

### Example 7

A highly-refractive liquid having a purity of 99.9 wt % or more, transmittance 99.2%/mm, and a refractive index of 1.64 at a wavelength of 193 nm was obtained by adding 1 part by weight of activated carbon to 10 parts by weight of commercial bicyclohexyl (manufactured by Aldrich co.: transmittance of 0%/mm), stirring the mixture for 24 hours at a room temperature, and adsorbing and filtering the stirred mixture at three times by using 0.5 parts by weight of alumina for front step and 2 parts by weight of Silica gel for rear step.

### Example 8

A highly-refractive liquid having a purity of 99.9 wt % or more, transmittance 98.2%/mm, and a refractive index of 1.64 at a wavelength of 193 nm was obtained by adding 1 part by weight of activated carbon to 10 parts by weight of commercial trans-decahydronaphthalene (manufactured by Tokyo Chemical Industry Co., Ltd.: transmittance of 0%/mm), stirring for 24 hours at room temperature, and adsorbed to and filtered the stirred mixture for three times by using 0.5 parts by weight of alumina for front step and 2 parts by weight of silica gel for rear step.

### Example 9

The bicyclohexyl (transmittance of 99.2%/mm) purified in Example 7 was inserted into a quarts cell, the quarts cell was sealed in the atmosphere of nitrogen,, and the quarts cell was irradiated by an ArF excimer laser (L5837 manufactured by Hamamatsu Photonics K.K.) with an energy condition larger than by two digits or more than about 10 mJ of the general exposure condition as a simulation (total energy of 6,000 mJ). The measured transmittance of the sample was reduced to 96.7%/mm. By adsorbing and filtering 10 parts by weight of the sample with a column using 1 part by weight of silica gel, the transmittance at 193 nm was restored to 99%/mm or more.

### Comparative Example 1

The commercial trans-decahydronaphthalene (manufactured by Tokyo Chemical Industry Co., Ltd.) having a purity of 99 wt % had transmittance of 0%/mm and the refractive index thereof could not be measured.

### Comparative Example 2

By adsorbing and filtering 10 parts by weight of commercial trans-decahydronaphthalene (transmittance 0.8%/mm) by the use of 1 part by weight of silica gel, a purity of 99.9 wt % or more, a refractive index of 1.64, and a transmittance of 65.1%/mm at 193 nm were obtained.

### Comparative Example 3

By adsorbing and filtering 10 part of commercial trans-decahydronaphthalene (transmittance 0.8%/mm) by the use of 1 part by weight of alumina, a purity of 99.9 wt % or more, a refractive index of 1.64, and a transmittance of 71.5%/mm at 193 nm were obtained.

### Comparative Example 4

By adsorbing and filtering 10 parts by weight of commercial trans-decahydronaphthalene (transmittance 0.8%/mm) by the use of 1 part by weight of activated carbon, a highly refractive liquid having a purity of 99 wt % or more, a refractive index of 1.64, and a transmittance of 69.8%/mm at 193 nm were obtained.

In Comparative Examples 2 to 4, the transmittance of the liquid could not be sufficiently enhanced, since the purity was enhanced by bringing the liquid into contact with only one kind of adsorbent.

On the contrary, in Examples 1 to 8, the transmittance was remarkably enhanced, since the purity was enhanced by bringing the liquid into contact with the plural adsorbents.

It could be seen from Example 9 that when the liquid purified by bringing the liquid into contact with the plural adsorbents is re-purified after the exposure, the contact of the liquid with one kind of adsorbent could enhance the transmittance.

1. A method of purifying a liquid for immersion exposure, wherein a saturated hydrocarbon compound is brought into contact with a first adsorbent and a second adsorbent to obtain a liquid for immersion exposure containing said saturated hydrocarbon compound with a purity of 99.5 wt % or more.

2.-15. (canceled)

16. The method according to claim 1, wherein said saturated hydrocarbon with a transmittance of 98%/mm or more at a wavelength of 193 nm is obtained.

17. The method according to claim 1, wherein said saturated hydrocarbon with a transmittance of 99%/mm or more at a wavelength of 193 nm is obtained.

18. The method according to claim 16, wherein said saturated hydrocarbon compound is bicyclohexyl.

19. The method according to claim 18, wherein said first adsorbent is silica gel, and said second adsorbent is composed of a different material from said first absorbent.

20. The method according to claim 16, wherein said saturated hydrocarbon is a trans-decahydronaphthalene.

21. The method according to claim 16, wherein said first adsorbent is activated carbon and said second adsorbent is silica gel or alumina.

22. The method according to claim 16, wherein said first adsorbent is silica gel and said second adsorbent is alumina.

23. The method according to claim 22, wherein the contact of said saturated hydrocarbon with said first adsorbent and said second adsorbent is repeated until said saturated hydrocarbon with a transmittance of 98%/mm or more at a wavelength of 193 nm is obtained.

24. The method according to claim 1, further comprising bringing said saturated hydrocarbon compound into contact with said second adsorbent at the same time as bringing said saturated hydrocarbon compound into contact with the first adsorbent.

25. The method according to claim 1, further comprising bringing said saturated hydrocarbon into contact with said second adsorbent after bringing said saturated hydrocarbon compound into contact with the first adsorbent.

26. The method according to claim 1, further comprising bringing said saturated hydrocarbon compound into contact with a third adsorbent or third to n^{th} (where n is an integer equal to or larger than 4) adsorbents.

27. The method according to claim 26, wherein said first adsorbent is activated carbon, said second adsorbent is silica gel, and said third adsorbent is alumina.

28. An immersion exposure method, wherein the liquid for immersion exposure obtained by the method according to claim 1 is filled in a space between a photosensitive material on a substrate and a projection lens and said photosensitive material is exposed thorough said liquid for immersion exposure.

29. An immersion exposure method comprising:
a step of degassing the liquid for immersion exposure obtained by the method according to claim 1 and then supplying the degassed liquid to a space between a photosensitive material on a substrate and a projection lens;
a step of exposing said photosensitive material through said liquid for immersion exposure;
a step of recovering said liquid for immersion exposure after said step of exposing the photosensitive material; and
a step of bringing said recovered liquid for immersion exposure into contact with at least one kind of adsorbent,
wherein said liquid for immersion exposure is circulated between said space and said adsorbent.

30. An immersion exposure method comprising:
a step of obtaining a liquid for immersion exposure by the method according to claim 1;
a step of degassing said liquid for immersion exposure and then supplying the degassed liquid to a space between a photosensitive material on a substrate and a projection lens;
a step of exposing said photosensitive material through said liquid for immersion exposure;
a step of recovering said liquid for immersion exposure after said step of exposing the photosensitive material; and
a step of bringing said recovered liquid for immersion exposure into contact again with said first adsorbent and said second adsorbent,
wherein said liquid for immersion exposure is circulated between said space and said first and second adsorbent.

31. A liquid for immersion exposure containing a saturated hydrocarbon compound with a purity of 99.5 wt % or more, obtained by the method according to claim 1.

32. The liquid for immersion exposure according to claim 31, wherein said saturated hydrocarbon compound has a linear or a branched chain structure and has a carbon number of 12 or more.

33. The liquid for immersion exposure according to claim 31, wherein said saturated hydrocarbon compound has a structure including a cyclic backbone and has a carbon number of 7 or more.

34. An immersion exposure method using the liquid for immersion exposure according to claim 31.
